(19) United States
(12) Patent Application Publication
(10) Pub. No.: US 2020/0222260 A1
(43) Pub. Date: Jul. 16, 2020

(54) Regenerati Incupod

(71) Applicant: Crystal Sylver, San Francisco, CA (US)

(72) Inventor: Crystal Sylver, San Francisco, CA (US)

(21) Appl. No.: 16/244,121

(22) Filed: Jan. 10, 2019

(51) Int. Cl. A61G 11/00; A41B 13/06; A61G 10/02; A61M 21/00; A61N 5/06; A61G 7/057; A61N 1/39

(52) U.S. Cl. CPC A61G 11/009; A41B 13/06; A61G 10/026; A61M 21/0094; A61N 5/06; A61G 7/05738; A61G 2200/14; A61M 2021/0027; A61N 2005/0659; A61N 2005/0663; A61M 2021/0022; A61M 2021/0066; A61G 2203/20; A61N 1/3904

(57) ABSTRACT

The invention is a regenerative pod for premature babies.

### BACKGROUND OF INVENTION

The Regenerati Incupod is a mechanical medical device. It is a regenerative premature baby incubator with several healing modalities, designed to replicate the mother's womb and aid in rapid healing.

The current models of baby incubators provide warmth and prevent heat loss but they do not offer any healing modalities suited to premature babies. Premature babies have more health related problems not only at birth, but also throughout their lives, costing the United States up to $50 billion a year.

### BRIEF SUMMARY OF INVENTION

The Regenerati Incupod is a baby incubator designed to renew, regenerate, and heal a premature baby while maintaining protection from outside elements that may shock the nervous system. The invention features light, sound, and oxygen therapies with a warming/vibrating gel waterbed and black tinted visor in order to replicate the mother's womb, the place where the premature baby should still be developing.

Current models are essentially a temperature controlled clear glass box in which a baby must wear protective eyewear because the bright light is harmful to underdeveloped eyes. A flat bed is provided with minimal support for fragile bodies. The loud noise of a hospital echoes through the baby, disrupting peace and calm, which is also required for healthy development. The current models do not offer built-in light, sound, or oxygen therapies.

### BRIEF DESCRIPTION OF THE SEVERAL VIEWS OF THE DRAWING

1/1 FIG. 1 Top view of Regenerati Incupod,

FIG. 2 Back view of Regenerati Incupod,

FIG. 3 Side view of Regenerati Incupod,

FIG. 4 Three-quarter view of Regenerati Incupod.

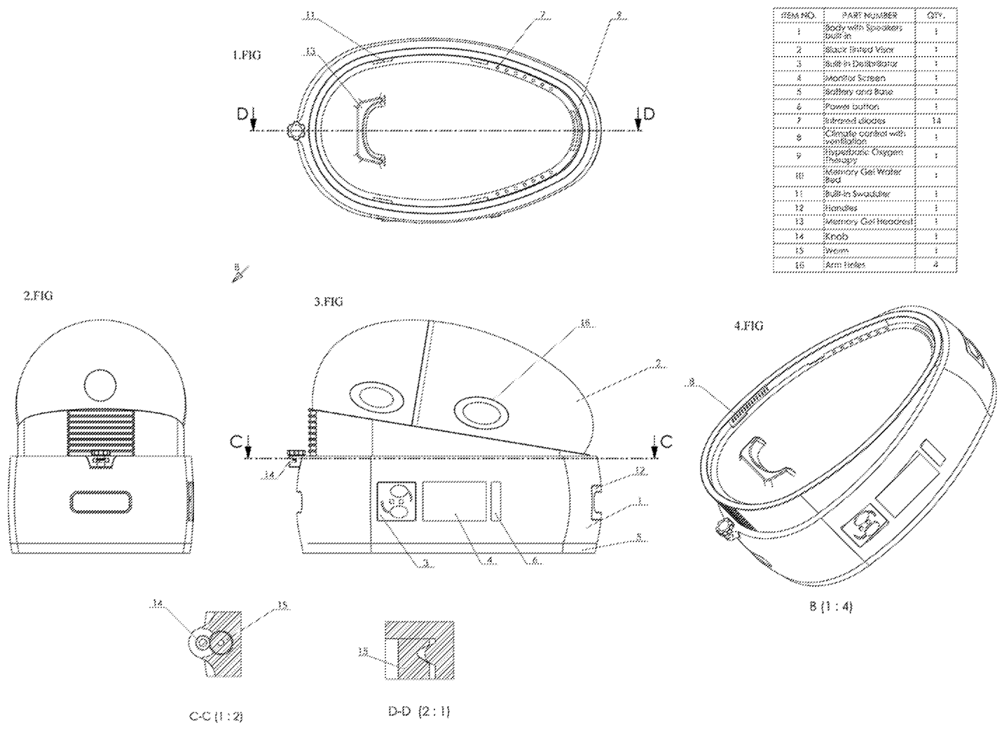

1. Body with Speakers built-in
2. Black Tinted Visor
3. Built-in Defibrillator
4. Monitor Screen
5. Battery and Base
6. Power Button
7. Infrared Diodes
8. Climate Control with Ventilation
9. Hyperbaric Oxygen Therapy
10. Memory Gel Water Bed
11. Built-in Swaddler
12. Handles
13. Memory Gel Headrest
14. Knob
15. Worm
16. Arm Holes

(1) Body of Regenerati Incupod with built-in sound speakers. (2) Black tinted visor encloses top and comes in two removable pieces. (3) Built-in defibrillator on side of body. (4) Monitor screen. (5) Battery and base of device. (6) Power button powers on and off. (7) Infrared and red light diodes (fourteen) inside chamber. (8) Climate control with ventilation inside chamber. (9) Hyperbaric oxygen therapy inside chamber. (10) Memory gel waterbed. (11) Built-in baby swaddler inside chamber. (12) Handles for portability. (13) Memory gel headrest with waterbed. (14) Knob for incline of device. (15) Worm for incline. (16) Arm holes on both sides of visor (four).

### DETAILED DESCRIPTION OF THE INVENTION

The Regenerati Incupod includes several new features and benefits that are not a part of standard incubators today. One feature is the black tinted visor on top of the body that comes in two detachable parts with the ability to see within the chamber (like a black tinted car window), which helps to shield a baby's sensitive eyes and skin from the harsh and bright lighting of hospitals. Currently hospitals use gauze or eye goggles over eyes because the glass surrounding the baby is clear. The eyes and skin of premature babies should still be in a pitch-black environment in order to develop properly.

Another feature is the memory gel water bed inside the chamber, which the baby lies on, with warming and vibrating waves to support a calm sleeping environment, similar to what a mother's womb would feel like, enclosing, warm, and in a constant state of movement with the heart beating and internal organs working, creating an environment of soft nurturing sound and motion that the baby should still be in. The memory gel water bed comes with a memory gel headrest to support the baby's fragile head, which also allows the baby to sleep in the fetal position, a much more natural position for the baby. The headrest can be molded and shaped to best suit the baby. The bed can be flat or raised to an incline position to support weightlessness and relieve pressure on the brain and bones. The knob and worm create a manually adjustable incline for the baby. The incubators today do not have any of these features.

The Regenerati Incupod's body has built-in speakers for sound healing. The vibrations of sound move through the incubator as well as the waterbed. Recorded sounds of the heart beating, the mother's voice, and soft music aid in healthy development and behavior attachments. Opposed to the baby listening to the often loud, frantic, and jarring noises of a hospital, the baby should be shut-off and protected from those noises, listening to and feeling calm sounds and vibrations. The device may also be soundproof.

Several powerful healing modalities of the Regenerati Incupod include infrared light therapy, red light therapy, hyperbaric oxygen therapy, and sound therapy. These are located within the chamber. The infrared and red light therapies are reflected and diffused through the chamber from the sides. These therapeutic methods can have enormous health benefits for a premature baby. Infrared light therapy promotes relaxation and calm energy, reduces anxiety and irritability, promotes a deeper, more restful sleep, soothes inflamed tissues, relieves headaches, sinus pressure and congestion, sore throats, ear aches, and coughing. Red light therapy speeds the healing of skin conditions, stimulates tissue and collagen growth, reduces inflammation and pain, and prevents hair loss and stimulates hair growth. Hyperbaric oxygen therapy helps fight bacteria and stimulates the release of growth factors and stem cells, which promote healing. Sound therapy promotes relaxation and reduces sleeping disorders, anxiety, depression, and pain.

The Regenerati Incupod also has a built-in baby swaddler to aid in the comfort and security of the premature baby. This feature secures the baby inside the pod through cloth wraps that can be changed daily, which pull out from each side of the bed. The cloth is wrapped around the baby, and is attached to the opposite side of the pod through an opening in the material, securing the baby in place. Current incubator models do not have this feature. The baby may also be held in a safe manner, protecting its fragile limbs, by taking the removable waterbed with swaddled baby out.

The Regenerati Incupod has a monitor screen built into the body which records heart rate, body temperature, climate, breathing patterns, and air purity, among other things. It also creates automation in the device for the incline if necessary, the vibration of the waterbed, and all sound, light, and oxygen healing modalities.

The pod has the ability to self-adjust in order to maintain proper temperature and climate for the baby. The built-in climate control with ventilation and air purifier inside the chamber can self-adjust to maintain the purity of the air and a hypoallergenic environment for the baby, controlling the temperature and air humidity, as well as filter dust and kill bacteria and germs.

Another feature is a built-in infant defibrillator next to the monitor screen in case of emergency cardiac arrest. And Four armholes with protective wrist guards, two on each side of the visor in order to minimize dust and allergens entering the incubator pod. There is a circular opening at the head and at the base of the visor, which allows for tubes into the chamber. This can also be sealed shut.

The base is also a large battery maintaining the power of the device. Handles on either side of the device allow for portability and movability.

The monitor screen can be located on either side of the device and can be any size. The defibrillator can be housed on either side of the device, or it may not be built-in. The power button can be located anywhere. The battery can be housed in another area inside the body rather than at the base and can be shaped differently. The device may or may not have handles built-in. The visor may be one piece, two pieces, three pieces, or more. It may or may not be tinted black. It may be plastic or glass. The armholes may not have protective wrist guards and they may be located anywhere that is most convenient for access to the baby. The Regenerati Incupod may be made any size, which is best suited for baby development. It may or may not have a built-in swaddler with changeable cloths. The infrared light diodes may or may not be built into the chamber. They may be above the baby or outside the device. They may also be built into any other area around the chamber. And there may be any number of diodes besides just seven on each side. The climate control with ventilation may be built into any area around the chamber. It may or may not be built into the chamber at all. The hyperbaric oxygen may or may not be built into the chamber. It may also be built into any area around the chamber. The device may or may not have an incline, which can be manually operated or automatic. The device may or may not have a gel waterbed. It may be a regular bed. It may or may not have a headrest. The bed may or may not have vibrating and warming elements. The body may or may not have speakers built-in. The device may be any shape, including rectangular, square, circular, or oval or egg shaped.

The Regenerati Incupod is designed for hospital use only.

The Regenerati Incupod can be manufactured in a facility that makes baby incubators or infrared light beds.

The Regenerati Incupod can be developed for premature babies, infants, children, teens, and adults for optimal healing environments. The invention may incorporate any number of healing modalities.

This invention improves upon the current model of baby incubator because it provides a nurturing, calming, and regenerative healing environment crucial to early development. It may also open up new ways of healing infants, children, teens, and adults.

1. The invention claimed is a Regenerati Incupod comprised of:
a portable body shaped like a pod with built-in sound speakers, two handles, one at each end, a monitor screen, power button, defibrillator, and knob for manual incline of the bed;
a black tinted rounded glass-like visor comprised of two removable pieces and four arm holes, two on each side, with protective wrist guards, and an opening at the head and at the base to allow for tubes into the chamber, which can also be sealed shut;
infrared light diodes, seven on each side built into the chamber, which are used for reflected and diffused infrared and red light therapy;
hyperbaric oxygen therapy built into the chamber;
climate control with ventilation built into the chamber;
a base and battery;
a removable memory gel waterbed with molded headrest that is vibrating and warming;
a built-in baby swaddler with changeable cloths.

2. The Regenerati Incupod as recited in claim 1, may house the monitor screen and power button on any area of the body. They may be of any size or shape.

3. The Regenerati Incupod as recited in claim 1, may not have a defibrillator built-in or it may be located anywhere convenient in the body.

4. The Regenerati Incupod as recited in claim 1, may house the battery in another area inside the body rather than at the base and it may be shaped differently to suit that area.

5. The Regenerati Incupod as recited in claim 1, may use an electrical plug rather than a battery.

6. The Regenerati Incupod as recited in claim 1, may not have handles built into each end for manual transporting.

7. The Regenerati Incupod as recited in claim 1, may not be portable.

8. The Regenerati Incupod as recited in claim 1, may use a visor that is not tinted black, but is clear, or another shade of grey, or colored.

9. The Regenerati Incupod as recited in claim 1, may be comprised of one piece, or three pieces, or four pieces, or more, rather than two separate pieces.

10. The Regenerati Incupod as recited in claim 1, may use a visor that is glass or plastic.

11. The Regenerati Incupod as recited in claim 1, may not use protective wrist guards in the armholes.

12. The Regenerati Incupod as recited in claim 1, may place the armholes anywhere in the visor that is most convenient for accessibility to the baby.

13. The Regenerati Incupod as recited in claim 1, may not have any openings in the visor. It may be fully enclosed.

14. The Regenerati Incupod as recited in claim 1, may be made rectangular, square, circular, oval, egg, or any number of shapes.

15. The Regenerati Incupod as recited in claim 1, may not have infrared or red light diodes built into the chamber. Or just one or the other. They may also be built above, into the visor, or anywhere else in the chamber. There may be any number of diodes or different shaped lights, such as rectangle.

16. The Regenerati Incupod as recited in claim 1, may have the climate control with ventilation built into any area around the chamber or it may not be built into the device at all.

17. The Regenerati Incupod as recited in claim 1, may have the hyperbaric oxygen therapy built into any area around the chamber or it may not be built into the device at all.

18. The Regenerati Incupod as recited in claim 1, may not have a manual or automatic incline. It may have a fixed incline or it may be flat.

19. The Regenerati Incupod as recited in claim 1, may not have sound speakers built-in.

20. The Regenerati Incupod as recited in claim 1, may be built for infants, children, teens, or adults.

21. The Regenerati Incupod as recited in claim 1, may not have a removable memory gel waterbed. It may be stationary. The device may use any other kind of bed or mattress besides a memory gel waterbed.

22. The Regenerati Incupod as recited in claim 1, may not have a vibrating or warming bed.

23. The Regenerati Incupod as recited in claim 1, may not have a headrest in the bed.

24. The Regenerati Incupod as recited in claim 1, may not have a built-in baby swaddler.
